(19) United States
(12) Patent Application Publication
(10) Pub. No.: US 2024/0366808 A1
(43) Pub. Date: Nov. 7, 2024

(54) METHOD FOR PREPARING AN [18F] RADIOLABELLED COMPOUND WITH LOW WATER CONTENT DURING LABELLING STEP

(71) Applicant: GE Healthcare Limited, Buckinghamshire (GB)

(72) Inventors: Torgrim ENGELL, Oslo (NO); Graeme MCROBBIE, Chalfont St. Giles (GB); Alan CLARKE, Oslo (NO); Julian GRIGG, Chalfont St. Giles (GB); Imtiaz Ahmed KHAN, Chalfont St. Giles (GB); Kristine WIKENE, Oslo (NO); Jonathan Robert SHALES, Chalfont St. Giles (GB); Alexander JACKSON, Bracknell (GB)

(21) Appl. No.: 18/569,180

(22) PCT Filed: Jun. 16, 2022
(86) PCT No.: PCT/EP2022/066483

§ 371 Date: Dec. 11, 2023

(30) Foreign Application Priority Data
Jun. 16, 2021 (GB) 2108605.3

(51) Int. Cl. A61K 51/04; C07B 59/00

(52) U.S. Cl. CPC A61K 51/0459; A61K 51/0453; A61K 51/0491; A61K 51/0493; C07B 59/002; C07B 59/004; C07B 59/005; A61K 2121/00

(57) ABSTRACT

The invention relates to a method of preparing an [¹⁸F] radio-labelled compound, wherein the water content is controlled. Controlling the water content and the origin of the water within the reaction process has a significant effect on both the yield and the purity of the product of the radio-labelling process.

### FIELD OF THE INVENTION

The present invention generally relates to a method of preparing a radio-labelled compound. It has been found that water content and origin of the water within the reaction process have a significant effect on both the yield and the purity of the product of the radio-labelling process.

### BACKGROUND

Radiopharmaceuticals are compounds labelled with a radioactive element, suitable for in vivo mammalian administration, for use in the field of medical imaging, diagnosis or therapy. Radiopharmaceutical compositions comprise a radio-labelled compound or a pharmaceutically acceptable salt thereof, solvent, and one or more stabilizers.

Fluorine-¹⁸ ([¹⁸F]) is a radioactive fluorine isotope commonly used in radiopharmaceuticals suitable for use in diagnosis. Fluorine-¹⁸ decay occurs by positron emission (97%) and electron capture (3%). As the radioisotope [¹⁸F] decays, the positrons emitted are utilised in positron emission tomography (PET) imaging. This in vivo imaging method is used, inter alia, in cardiac imaging, tumour imaging and brain imaging.

Automated synthesis systems are important for the production of radiopharmaceuticals. Synthesis modules of the prior art are described in WO 2007/042781 and WO 2011/097649. Synthesis modules, such as the FASTlab® (GE Healthcare) provide for production of doses of radiopharmaceuticals for clinical applications. The FASTlab synthesis module accepts and operates a method through a device for producing a radiopharmaceutical.

In processes preparing radiolabelled compounds, radiochemical impurities and unreacted [¹⁸F] fluoride are undesirable by-products. It would be advantageous to minimise these by-products.

### SUMMARY OF THE INVENTION

The present invention relates to an improved method for preparing an [¹⁸F] fluoride radiolabelled compound.

An aspect of the invention relates to a method of preparing an [¹⁸F] radiolabelled compound, wherein the method comprises
(a) an initial drying step comprising evaporating water and acetonitrile from a solution comprising [¹⁸F] fluoride;
(b) a further drying step (fluoride activation drying step), comprising azeotropic distillation of water from said solution comprising [¹⁸F] fluoride with acetonitrile; and
(c) labelling a precursor compound with [¹⁸F] fluoride from the solution comprising [¹⁸F] fluoride yielded from step (b) to obtain an [¹⁸F] radiolabelled compound;
wherein the water content during labelling step (c) that originates from the solution comprising [¹⁸F] after drying step (b) is less than 500 ppm; and
wherein the water content during labelling step (c) that originates from the precursor compound is no more than 2000 ppm.

Preferably at least two cycles of the azeotropic distillation with acetonitrile are carried out. More preferably, three cycles of azeotropic distillation with acetonitrile are carried out.

In an aspect of the invention, the water content during the labelling step that originates from the solution comprising [¹⁸F] after drying step (b) is less than 400 ppm. Preferably, the water content during the labelling step that originates from the solution comprising [¹⁸F] after drying step (b) is less than 350 ppm. In an aspect of the invention, the water content during the labelling step that originates from the precursor compound is no more than 1500 ppm. Preferably, the water content during the labelling step that originates from the precursor compound is between 500 ppm and 1000 ppm. In an aspect of the invention, the total water content during the labelling step is less than 2500 ppm, for example, less than 1000 ppm.

In another aspect of the invention, the radioactivity of [¹⁸F] prior to step (a) (at the start of synthesis) is up to around 500 GBq, for example up to around 450 GBq, up to around 400 GBq, up to around 350 GBq, up to around 300 GBq, or for example between 50 GBq and 250 GBq. By using the process of the invention, a high yield of the fluorinated product and a low amount of radiochemical impurity are obtained, even when the radioactivity of [¹⁸F] fluoride at the start of synthesis in the process of the invention (the starting activity) is greater than 100 GBq. The ability to use a higher starting activity and still achieve a high yield and low amount of radiochemical impurity enables a greater number of product doses to be prepared in a single batch.

In another aspect of the invention, the radiolabelled compound is a [¹⁸F] fluoride-labelled radiopharmaceutical, or a pharmaceutically acceptable salt thereof.

### FIGURES

FIG. 1 is a flow chart of the current process steps for preparing a radiolabelled product.

FIG. 2 is a flow chart of the process of the invention for preparing a radiolabelled product.

### DETAILED DESCRIPTION OF THE INVENTION

The term ‘radiopharmaceutical’ has its conventional meaning and refers to a radioactive compound suitable for in vivo mammalian administration for use in diagnosis or therapy. A radiopharmaceutical as referenced herein may be a Positron Emission Tomography (PET) tracer.

Before a radiopharmaceutical composition, or ‘drug product’ can be administered to a patient, it must undergo a thorough quality control (QC) process, to ensure that it complies with requirements, such as purity.

Radiochemical purity (RCP) is determined using radio TLC or HPLC and can be defined as the ratio of the (radio-labelled) drug substance peak to the total (radio-labelled) peaks in the chromatogram. If one manufactures a radiopharmaceutical with high radioactive concentration (RAC), the drop in RCP during storage is likely to be higher than at lower RAC due to increased radiolysis. High radioactive concentration results in the drug substance destroying itself (i.e. radiolysis).

The term ‘comprising’ has its conventional meaning throughout this application and implies that the method, system, product or the like must have the components listed, but that other, unspecified components may be present in addition.

A radio-labelled compound may comprise various radio-isotopes. For example, the radio-labelled compound may be a ¹⁸F-labelled radiopharmaceutical, or a pharmaceutically acceptable salt thereof. The radio-labelled compound may be: an ¹⁸F-labelled radiopharmaceutical, or a pharmaceutically acceptable salt thereof.

The radio-labelled compound may be a ¹⁸F-labelled radiopharmaceutical, or a pharmaceutically acceptable salt thereof. Examples of such ¹⁸F-labelled radiopharmaceuticals include [¹⁸F] FDG (2-deoxy-2-[¹⁸F] fluoro-D-glucose), [¹⁸F] FMAU (2′-deoxy-2′-[¹⁸F] fluoro-5-methyl-1-beta-D-arabinofuranosyluracil), [¹⁸F] FMISO (¹⁸F Fluoromisonidazole), [¹⁸F] FHBG (9-(4-[¹⁸F] Fluoro-3-[hydroxymethyl] butyl) guanine), [¹⁸F] FES (16a-[¹⁸F] fluoro-17b-estradiol) [¹⁸F] AV-45, [¹⁸F] AV-19, [¹⁸F] AV-1, [¹⁸F] Flutemetamol, [¹⁸F] Flurpiridaz, [¹⁸F] K5, [¹⁸F] HX4, [¹⁸F] W372, [¹⁸F] VM4-037, [¹⁸F] CP¹⁸, [¹⁸F] ML-10, [¹⁸F] T808, [¹⁸F] T807, 2-[¹⁸F] fluoromethyl-L-phenylalanine, GE-135 [¹⁸F] Fluciclatide, GE-212, GE-226, or combinations thereof.

The radio-labelled compound may be a compound of Formula (I):

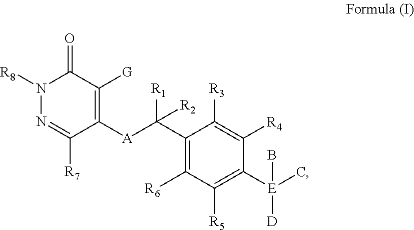

wherein A is selected from N(R⁷), S, O, C(═O), C(═O) O, NHCH₂CH₂O, a bond, or C(═O)N(R⁷);
when present, B is selected from hydrogen, alkoxyalkyl, alkyloxy, aryl, C₁-C₆ alkyl optionally substituted with an imaging moiety, heteroaryl, and an imaging moiety;
when present, C is selected from hydrogen, alkoxyalkyl, alkyloxy, aryl, C₁-C₆ alkyl optionally substituted with an imaging moiety, heteroaryl, and an imaging moiety;
D is selected from hydrogen, alkoxyalkyl, alkyloxy, aryl, C₁-C₆ alkyl optionally substituted with an imaging moiety, heteroaryl, and an imaging moiety; or
C and D, together with the atom to which they are attached, form a three- or four-membered carbocyclic ring;
G is halo or haloalkyl;
n is 0, 1, 2, or 3;
R¹, R², R³, R⁴, R⁵, R⁶ and R⁷ are independently selected from hydrogen, C₁-C₆ alkyl optionally substituted with an imaging moiety, and an imaging moiety;
R⁸ is C₁-C₆ alkyl, optionally substituted with an imaging moiety; and E is selected from a bond, carbon, and oxygen, provided that when E is a bond, B and C are absent and D is selected from aryl and heteroaryl, and provided that when E is oxygen, B and C are absent and D is selected from hydrogen, alkoxyalkyl, aryl, C₁-C₆ alkyl optionally substituted with an imaging moiety, and heteroaryl;
provided that at least one imaging moiety is present in Formula (I).

Substituent A of Formula (I) may be O. R⁸ may be tert-butyl. G may be chloro. The imaging moiety may be any radio-isotope as referenced herein, for example [¹⁸F].

The radio-labelled compound may be [¹⁸F] flurpiridaz, which has the following structure:

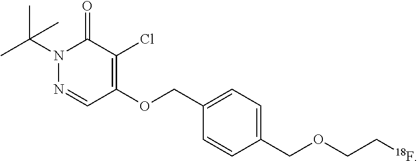

### Description of the Production Process

The method of the present invention may be carried out on an automated synthesis system, such as the FASTlab® system (GE Healthcare) that provides for production of doses of radiopharmaceuticals for clinical applications.

In the description below, the FASTlab® system is referred to, however this is not limiting on the present invention and another suitable system may be used.

The term [¹⁸F] is used covering both the non-ionic and the anionic form. [¹⁸F] fluorine is in anionic form and hence the term [¹⁸F] fluoride is commonly used. The scale of an [¹⁸F] PET tracer manufacture is measured in radioactivity (‘activity’) used at the start of synthesis (‘SOS’), also referred to herein as the ‘starting activity’ or ‘starting radioactivity’. An activity of 100 GBq equals 14.2 ng [¹⁸F]. Generally, the higher the radioactivity, the greater the degree of radiolysis.

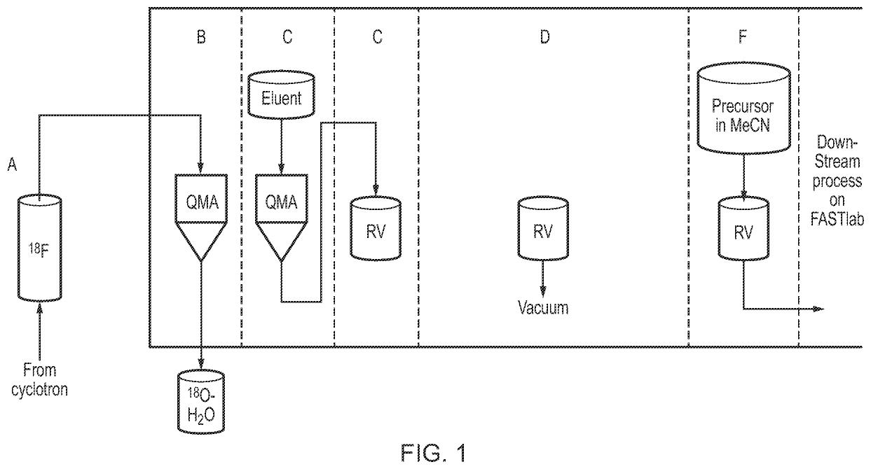

FIG. 1 shows a flowchart of part of the production process of a radiopharmaceutical.

In step A, [¹⁸F] fluoride is produced using a GE Medical Systems PETtrace cyclotron with a silver target via the [¹⁸O] (p,n) [¹⁸F] nuclear reaction. Total target volumes of 3 to 5 mL are used. In step B, [¹⁸F] can be transferred from interim storage, or directly from a cyclotron, onto the FASTlab system, or another suitable system. The use of interim storage is preferred, in order to be able to measure and control the amount of radioactivity to be transferred onto the FASTlab. When transferred onto the FASTlab, [¹⁸F] is trapped on an anionic solid phase extraction (SPE) cartridge, e.g. QMA cartridge (pre-conditioned with carbonate) (Waters Corporation). The activity transferred onto FASTlab is also measured in-line by a calibrated radio detector placed behind the QMA cartridge. In step C, the [¹⁸F] is eluted off the QMA cartridge, for example, with a solution of tetrabutylammonium hydrogen carbonate in water and acetonitrile (e.g. 400 μL). Nitrogen was used to drive the solution off the QMA cartridge and transferred to the FASTlab reactor (reaction vessel, RV). In step D, initial evaporation of water and acetonitrile takes place at elevated temperature, e.g. 120° C., under a steady stream of nitrogen and under vacuum. In step F, the compound to be radiolabelled (also referred to herein as the ‘precursor’, or ‘final intermediate’), dissolved in acetonitrile, is added to the reaction vessel. The precursor may for example carry a tosyl group (tosylate) that will be replaced by the ¹⁸F-radiolabel. This fluorination step yields the crude product. Subsequently, purification steps are carried out to yield the pure radiolabelled compound (pure drug substance) and, following sterile filtration, the drug product.

Several experiments were performed to investigate the impact that increasing levels of water in the precursor vial had on the radiolabelling process.

The water content during the radiolabelling reaction in step F was found to be an important variable in the amount of radio-impurity (for example, radiochemical impurity B, depicted below) formed in the crude product.

The structure of radioimpurity B is as follows:

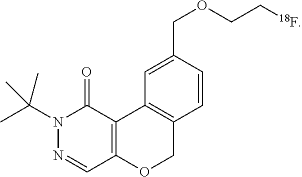

Experimental results support the hypothesis that radiochemical impurities (for example, radiochemical impurity B) are formed via a free radical radiolysis mechanism. Water is a potential source of free radicals and a high amount of analogue of the hydroxy impurity was observed in the LC-MS analysis of the crude product. The inventors believe that the relationship between the amount of free or hydroxy radicals formed during the drying (step D) and the water content present during the labelling reaction is key. More free radicals are generated during the drying process due to the higher RAC, the higher temperature and longer process time. The inventors have determined that water needs to be minimised during this part of the process, in order to supress the formation of free radicals, including hydroxy free radicals.

The water content during the radiolabelling step (step F) is composed of the following: a) water carried over from the drying step, and b) water in the vial containing the precursor from the solid material and the acetonitrile used for dissolution. It has been determined that the improved drying process of the present invention reduces the number of free radicals entering the labelling reaction.

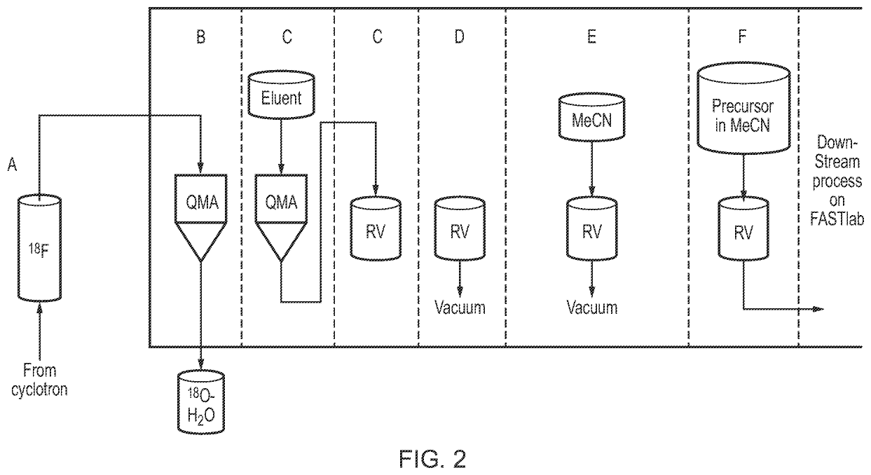

FIG. 2 shows a flowchart of the process comprising the additional process steps of the present invention. Steps A to D and F are as described in relation to FIG. 1, above. In new step E (following on from step D) an additional drying procedure is carried out, also referred to herein as the fluoride activation (drying) step. The drying procedure of the solution comprising [¹⁸F] in step E includes azeotropic distillation of water/acetonitrile, by addition of acetonitrile followed by evaporation at elevated temperature under vacuum. In the enhanced drying procedure of the invention this step is repeated at least two times. Preferably three azeotropic drying cycles (3×0.5 mL acetonitrile) are carried out. Step E is followed by step F, the fluorination (radiolabeling) step described above in relation to FIG. 1.

The water content of the radiolabelling reaction was investigated via a series of non-radioactive experiments using a Karl Fischer apparatus to measure the water content. Three or four samples were analysed for each experiment summarised in Table 1: The water content of (i) the acetonitrile used to dissolve the precursor, (ii) the acetonitrile used for the azeotropic drying, (iii) the dissolved precursor, (iv) carried over from the drying process and (v) the labelling solution itself, were analysed.

TABLE 1
Summary of water content in fluoride drying experiments

| | Water content (ppm) | | | | |
|---|---|---|---|---|---|
| | | In acetonitrile | | | |
| | In acetonitrile | used for | | Carried over | |
| Experiment | used to dissolve | azeotropic | In dissolved | from drying | Total during |
| # | precursor | drying | precursor | (calculated) | labelling step |
| 1 | 60 | n/a | 231 | 2238 | 2469 |
| 2 | 60 | n/a | 274 | 2391 | 2665 |
| 3 | 60 | 60 | 230 | 375 | 605 |
| 4 | 80 | 2016 | 251 | 311 | 562 |
| 5 | 63 | 63 | 282 | 323 | 605 |
| 6 | 60 | n/a | 222 | 933 | 1155 |
| 7 | 60 | 60 | 302 | 416 | 718 |

### Experiments 1, 2 and 6 are Reference Examples

The structure of the precursor is as follows:

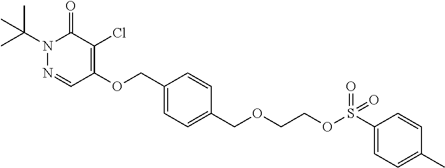 [SMILES: Cc1ccc(S(=O)(=O)OCCOCc2ccc(COc3cnn(C(C)(C)C)c(=O)c3Cl)cc2)cc1]

This precursor is particularly susceptible to radiolysis degradation.

The total water content during the labelling reaction (last column) is made up of the water content originating from the precursor vial and the water content present in the solution comprising [¹⁸F] fluoride after the drying process.

The water content in the initial high activity experiments was about 2500 ppm (Table 1, Experiment 1). The drying regime for this sequence was about 8.5 minutes at 120° C. (‘original sequence’).

In Experiment 2 a widely-used drying sequence is applied. Compared to the drying sequence of Experiment 1, in Experiment 2 the temperature is held at 120° C. for about an extra 1.5 minutes with a slight difference in the inert gas flow rate to the reaction vessel during the early evaporation steps. The total drying time was thus about 10 minutes. The longer drying time had no significant effect on the water content during the labelling reaction, the water content in Experiment 2 being 2665 ppm compared to 2469 ppm in Experiment 1. Further drying sequences detailed below are based on the drying sequence described for Experiment 2.

In Experiment 3 and 4, azeotropic drying (3×0.5 mL acetonitrile) was added to the drying sequence of Experiment 2. The total drying time was about 15 minutes at 120° C., meaning that the addition of three azeotropic drying cycles added about 5 to 6 minutes to the total drying time. In Experiment 3 the water content during labelling was determined to be 605 ppm, of which 375 ppm was water carried over from the drying step and 230 ppm originated from the precursor vial.

In Experiment 5 an alternative azeotropic drying sequence was developed at 110° C. instead of 120° C. with a lower vacuum set point and three azeotropic drying cycles (3×0.5 mL acetonitrile). The total drying time was 12.8 minutes. This sequence has the advantage that there is no need for a reduction in temperature (cooling step) before the precursor is added to the reaction vessel. Furthermore, the total drying time is shorter than the drying sequence in Experiments 3 and 4 (12.8 minutes vs 15 minutes). The water content was 605 ppm, which was the same as in the sequence with the harsher conditions (Experiment 3). The enhanced drying procedure of Experiment 5 reduced the water content in the solution comprising the [¹⁸F] component after drying from 2238 ppm to 323 ppm (compare Experiments 1 and 5).

It was also postulated that rinsing the QMA cartridge with acetonitrile after the [¹⁸F] fluoride was trapped (and before it was eluted into the reaction vessel) would reduce the amount of water going into the reaction vessel as the residual water on the QMA cartridge would be replaced with acetonitrile. Therefore, in Experiment 6, a rinse of the QMA cartridge with acetonitrile via syringe S1 was carried out and the [¹⁸F] fluoride was dried using the drying process of Experiment 2. The amount of water in the labelling reaction in Experiment 6 was 1155 ppm, which suggests the rinse of the QMA cartridge results in an improvement (that is, a reduction) in the water content, compared to the simple drying procedure used in Experiment 2. However, in Experiment 7, which combines the azeotropic drying sequence of Experiments 3 and 4 with the QMA cartridge rinse, the water content was measured to be 718 ppm, which is higher than without the rinse (compare Experiments 6 and 7). This demonstrated that the acetonitrile rinse of the QMA is unnecessary when the solution comprising the [¹⁸F] fluoride is azeotropically dried.

In conclusion, the enhanced [¹⁸F] fluoride drying process of Experiment 5 involved azeotropically drying with 3 portions of acetonitrile (the fluoride activation drying step). The total drying time was just under 13 minutes at 110° C., which is also the temperature required for the subsequent labelling step.

Preferably at least two cycles of the azeotropic distillation with acetonitrile are carried out. More preferably at least three cycles of the azeotropic distillation with acetonitrile may be carried out. Most preferably, three cycles of azeotropic distillation with acetonitrile are carried out.

Preferably the water content during the radiolabelling step is less than 1000 ppm. More preferably, the water content during the radiolabelling step is less than 700 ppm.

Preferably the water content during the radiolabelling step originating from the solution comprising [¹⁸F] after the drying steps (including the fluoride activation drying step) is less than 500 ppm. More preferably, the water content during the radiolabelling step originating from the solution comprising [¹⁸F] after the drying steps (including the fluoride activation drying step) is less than 400 ppm. Even more preferably, the water content during the radiolabelling step originating from the solution comprising [¹⁸F] after the drying steps (including the fluoride activation drying step) is less than 350 ppm.

The enhanced drying step is also believed to lead to the removal or reduction of the water molecules associated with fluoride ions, enabling the fluoride to more easily participate in the radiolabelling reaction. That is, by liberating the fluoride from its solvent cage of water molecules, the fluoride becomes available for reaction with an electrophile, e.g. forming a complex with Kryptofix-222 (Sigma Aldrich; Merck KGaA, Germany) or a tetrabutylammonium salt (ABX advanced biochemical compounds GmbH, Germany). This drying step may also be referred to herein as a ‘fluoride activation step’, or a ‘fluoride activation drying step’.

The reduced water content after drying (as described, for example, with respect to step E in FIG. 2) enables the radiolabeling step to be optimized. The major radio-chemical impurity is reduced to a level which enables an efficient purification using SPE to reduce the amount of this impurity to below specification limit of 2% relative to product. This is the case even at a starting radioactivity up to 350 GBq. As referred to above, higher radioactivity levels lead to higher levels of radiolysis and therefore radiochemical impurities. In other words, the higher the radioactivity, the greater the effects that are to be negated. At a radioactivity level of 350 GBq the impurity was expected to be obtained at a level even higher than the target compound. This would decrease the radio chemical yield and lower the number of drug product doses yielded to a non-acceptable level, regardless of which product purification method would be used. However, by using the process of the invention, a high yield of the fluorinated product and a low amount of radiochemical impurity were obtained. This high yield of the fluorinated product and a low amount of radiochemical impurity B are obtained even when the radioactivity of [¹⁸F] fluoride at the start of synthesis in the process of the invention (the starting activity) is greater than 100 GBq. The ability to use a higher starting activity and still achieve a high yield and low amount of radiochemical impurity enables a greater number of product doses (also referred to as ‘patient doses’) to be prepared from a single batch. At a starting activity of 250 GBq, more than 20 patient doses can be prepared from a single batch. The radioactivity of [¹⁸F] fluoride at the start of synthesis in the process of the invention (the starting activity) may be up to around 500 GBq, for example up to around 450 GBq, up to around 400 GBq, up to around 350 GBq, up to around 300 GBq, or for example, at least 100 GBq, between 50 GBq and 250 GBq, 100 GBq to 350 GBq, 200 GBq to 300 GBq, 200 GBq to 350 GBq, or 250 GBq to 350 GBq.

The amount of radioimpurity B in the product obtained by the process of the present invention is less than 3.5%, for example less than 3%, less than 2.5%, less than 2%, or less than 1.5%.

### Water Content of the Acetonitrile Vial

To determine the effect of the water content of the 100% acetonitrile vial used in the azeotropic drying process, the enhanced drying process of the invention (that is, including the fluoride activation drying step) was carried out with acetonitrile having two different water content levels. There are two shelf-life water content specifications for this vial, 750 ppm and 2000 ppm. A comparison of the water content carried over into the radiolabelling step from the azeotropic drying carried out with an acetonitrile vial containing 60 ppm or 2016 ppm water is summarised in Table 2.

TABLE 2
Water content carried over into the labelling reaction
after the drying step, with different amounts of water
in the acetonitrile vial used for azeotropic drying

| | Water | Water content | |
|---|---|---|---|
| | content | carried over as | |
| | of the | part of the solution | |
| | acetonitrile | comprising [¹⁸F]fluoride | |
| Experiment | vial | after the drying step | See also |
| 8 | 60 | 375 | Table 1, |
| | | | Experiment 3 |
| 9 | 2016 | 311 | Table 1, |
| | | | Experiment 4 |

There was no significant difference in the water content carried over from the drying step when using acetonitrile containing either 60 or 2016 ppm water in the azeotropic drying. Accordingly, the inventors found that water added at this stage of the process did not affect the end result.

High Activity Testing with Enhanced Drying Process

Table 3 compares the results of the radiolabelling step yielding the crude product, using the fluoride drying process of the present invention and the standard drying process.

[¹⁸F] fluoride was produced using a GE Medical Systems PETtrace cyclotron with a silver target via the [¹⁸O] (p,n) [¹⁸F] nuclear reaction. Total target volumes of 3 to 5 mL were used. The radiofluoride was trapped on a Waters QMA cartridge (pre-conditioned with carbonate), and the fluoride was eluted with a solution of tetrabutylammnonium hydrogen carbonate (22.8 mg) in water (100 μL) and acetonitrile (400 μL). Nitrogen was used to drive the solution off the QMA cartridge into the reaction vessel. The [¹⁸F] fluoride was dried for ca. 20 minutes at 110° C., including 3×0.5 mL acetonitrile azeotropic drying steps, under a steady stream of nitrogen and vacuum. The precursor (10.2 mg) in acetonitrile (1.7 mL) was added to the dried [¹⁸F] fluoride and the reaction mixture was heated at 110° C. for 3 minutes.

The radiolabelling was significantly improved with the drying process of the present invention: the yield of the crude radiolabelled product increased from 72% to 81%, the amount of unreacted [¹⁸F] fluoride decreased from 5% to 1% and the amount of radiochemical impurity B decreased from 22% to 13%.

TABLE 3
Summary of initial high activity experiments in acetonitrile

| | | | | | | | | Crude | |
|---|---|---|---|---|---|---|---|---|---|
| | Starting | | | | | | | Product | Radio- |
| | Activity | Precursor | Drying | TBA•HCO₃ | Time | Temperature | [¹⁸F]fluoride | (%) | impurity B |
| Experiment | (GBq) | (mg) | Sequence | (mg) | (min) | (° C.) | (%) | ([¹⁸F]Flurpiridaz) | (%) |
| 10 | 93 | 10.2 | Original - | 22.8 | 10 | 120 | 5 | 72 | 22 |
| | | | see Table 1, | | | | | | |
| | | | Experiment 2 | | | | | | |
| 11 | 100 | 10.2 | Optimised - | 22.8 | 10 | 120 | 1 | 81 | 13 |
| | | | see Table 1, | | | | | | |
| | | | Experiment 5 | | | | | | |

Experiment 10 is a reference example.

These results demonstrate the significant advantages achieved by the drying process of the present invention.

Confirmation of water content range of the precursor in acetonitrile 6 mg/mL vial The water content carried over from the drying step into the radiolabelling step was found to be an important variable in the amount of radiochemical impurity B formed in the crude product. Using the drying procedure of the present invention, the water content during the radiolabelling reaction is about 600 ppm (see Table 1). However, this provides a problem for the shelf-life of the precursor vial. The water content of the acetonitrile used to dissolve the precursor is 500 ppm and the water content of the precursor vial is expected to increase by ca. 15 ppm per month, due to natural water ingress into the vial over time. This would limit the shelf-life of the precursor vial to less than 7 months (i.e. (600-500)/15 ppm=6.7 months) to remain at the low water content desired in the labelling reaction. Ideally, the shelf-life of the precursor vial would be a minimum of 24 months. After 24 months, the precursor dissolved in acetonitrile would have a water content of >860 ppm (i.e. 15 ppm*24 months+500 ppm). Therefore, the radiolabelling process was challenged with higher levels of water in the precursor vial. Water was added to the precursor vial and the water content was measured using Karl Fischer apparatus. Results of these experiments are shown in Table 4. For each of experiments 12-18, the optimised drying sequence of experiment 5 in Table 1 was used (i.e. the water content carried over from the drying sequence is less than 500 ppm).

TABLE 4
Summary of FASTlab syntheses after SPE purification with increasing
levels of water in the precursor vial during SPE development work

| | Water | | | Non-decay | | | |
|---|---|---|---|---|---|---|---|
| | content of | | | corrected | | | |
| | precursor | | Starting | yield | Radio- | Radio- | |
| | vial | Drying | Activity | (NDCY) | impurity A | impurity B | [¹⁸F]Flurpiridaz) |
| Experiment | (ppm) | Sequence | (GBq) | (%) | (%) | (%) | (%) |
| 12 | 250 | Optimised - | 146 | 43 | 0.2 | 0.7 | 98.7 |
| 13 | 250 | see Table 1, | 250 | 33 | 0.3 | 2.7 | 96.7 |
| 14 | 250 | Experiment 5 | 246 | 33 | 0.4 | 1.6 | 97.4 |
| 15 | 1300 | | 132 | 38 | 0.2 | 1.3 | 97.9 |
| 16 | 1300 | | 239 | 31 | 0.3 | 1.8 | 97.7 |
| 17 | 2000 | | 249 | 33 | 0.3 | 1.8 | 97.5 |
| 18 | 4600 | | 194 | 20 | 0.4 | 1.5 | 97.8 |

Experiments 12 to 18 use the optimised drying process of the present invention.

Radiochemical yield refers to the total amount of radioactivity that is obtained after purification related to the starting radioactivity amount (e.g. obtained from a cyclotron or previous reaction step). Radiochemical yield is noted to be either decay corrected, or non-decay corrected (NDCY).

A lower yield and higher amount of radiochemical impurity B would be expected with a higher water content. However, surprisingly, the results in Table 4 show that increasing the water content of the precursor vial from 250 ppm to 2000 ppm had no observable impact on the radiolabelling process. This was a very surprising result and indicates that during the radiolabelling reaction, the water present in the precursor vial behaves differently to the water from the eluent solution comprising the [¹⁸F] fluoride after drying.

Table 5 shows the comparison of two experiments showing the improved results with the optimised drying sequence even with ca. 2 times higher starting activity. The SPE purified product has a higher purity and lower amount of the radioimpurity B.

TABLE 5
Comparison of SPE purified product using the original and optimised drying sequences.

| | Water content (ppm) | | | | | | | | |
|---|---|---|---|---|---|---|---|---|---|
| | In | In | | | | | | | |
| | acetonitrile | acetonitrile | | Carried | Total | | | | |
| | used to | used for | In | over from | during | Starting | | | Radio- |
| | dissolve | azeotropic | dissolved | drying | labelling | Activity | Drying | [¹⁸F]Flurpiridaz | impurity B |
| Experiment | precursor | drying | precursor | (calculated) | step | (GBq) | Sequence | (%) | (%) |
| 17 | 63* | 63* | 2000 | 323* | 2323 | 249 | Optimised - | 98 | 1.5 |
| | | | | | | | see Table 1, | | |
| | | | | | | | Experiment 5 | | |
| 19 | 63* | n/a | 274* | 2391* | 2665* | 125 | Original - | 90 | 5.6 |
| | | | | | | | see Table 1, | | |
| | | | | | | | Experiment 2 | | |

*Values not measured but are based on Experiments 2 and 5, Table 1

In order to meet the Product Specification, one of the requirements is that the amount of radioimpurity B must be below 3.5%. It can be seen that using the original process, without the optimised drying according to the present invention, the amount of radioimpurity B is 5.6%, when using a starting radioactivity of 125 GBq. In contrast, when using the optimised process of the present invention, the amount of radioimpurity B is 1.5%, even when using a much higher starting radioactivity of 249 GBq. In other words, the present invention enables the reaction to be scaled up more than two- or three-fold.

The effect of the starting radioactivity is further illustrated in Table 6, below:

TABLE 6
Results of experiments with >300 GBq starting activity (45 mL product volume)

| | Starting | Non-decay | | | |
|---|---|---|---|---|---|
| | Activity | corrected | RAC | [¹⁸F]Flurpiridaz | Radioimpurity B |
| Experiment | (GBq) | yield (%) | (MBq/mL) | at t = 0 (%)^{a} | at t = 0 (%)^{b} |
| 20 | 301 | 31.5 | 2104 | 97.2 | 1.9 |
| 21 | 344 | 35.2 | 2689 | 97.3 | 1.7 |
| 22 | 345 | 29.8 | 2282 | 98.1 | 1.0 |

^{a}Specification NLT 95%;
^{b}Specification NMT 3.5%;
Formulation: 30 to 32 mg/mL ascorbic acid, 40 mg/mL HP-β-CD

The experiments in Table 6, carried out according using the optimised drying process of the present invention, all have a starting activity above 300 GBq, and achieve a high product purity (RCP) (above 97%) and a low amount of radioimpurity B (less than 2%). This falls within the Product Specification requirements for the product.

In contrast, as demonstrated by Experiment 19 in Table 5, using the original drying process only achieved a lower RCP (90%) and an unacceptably high amount of radioimpurity B (5.6%). The original process would require a much lower starting activity in order to yield less radioimpurity B (less than 3.5% in order to meet product specification requirements), which in turn would result in the production of fewer patient doses per batch.

Accordingly, the optimised drying process of the present invention enables the use of a higher starting activity, producing a greater number of patient doses per batch with a high yield, high RCP and low amount of radioimpurity B.

An explanation can be found in the relationship between the amount of free or hydroxy radicals formed during the drying process and the water content present during the labelling reaction. More free radicals are generated during the drying process due to the higher RAC, the higher temperature and longer process time. The inventors believe that the improved drying process of the present invention reduces the number of free radicals entering the labelling reaction.

In summary, these results confirm that the water content specification of the precursor vial can be up to 2000 ppm which should be enough to provide a good shelf-life (2000 ppm-500 ppm/15=100 months, or over 8 years). As well as a longer shelf life, this tolerance for a higher water content of the precursor vial allows for easier manufacture of the vials of precursor.

The present invention shows that the water present in the FASTlab process behaves differently depending on when it is introduced to the process. The water added at the start of the process has more of an effect on the process than the water introduced in the radiolabelling step (i.e. residual water present from the precursor and solvent used to dissolve it).

The trapped [¹⁸F] fluoride is liberated from the ion exchange resin with acetonitrile and water. The eluent comprises [¹⁸F], water and acetonitrile. Due to the high radioactivity levels, if water is not removed prior to the radiolabelling (fluorination) step, a higher number of hydroxy free radicals are present. Current methods evaporate some water and acetonitrile prior to the radiolabelling step. However, it has been found that if the [¹⁸F] eluent (the solution comprising [¹⁸F]) is dried in advance of the radiolabelling step, according to the enhanced drying step of the present invention, the presence of fewer free radicals leads to a higher yield of a purer product with fewer radiochemical impurities present. The lower the amount of residual water from the [¹⁸F] eluent remains when carrying out the radiolabelling step, the fewer radioimpurities are generated in the radiolabelling reaction step. The effect of the presence of the hydroxy free radicals of the water in the [¹⁸F] eluent is reduced or avoided.

The subsequent addition of new water that has not been exposed to radioactivity/radiolysis into the radiolabelling step does not have a negative impact on the yield of the crude product or increase the impurities present.

The present invention also demonstrates that the origin of the water content in FASTlab processes is more important in earlier steps than later. Water that is still present after fluoride drying has been found to have a greater impact than water present in the precursor solution. This was unexpected as it was expected that water behaves the same way at each step. A further advantage of the present invention is that the increased water content of the precursor vial allows for a longer shelf-life of the vial. This also enables a greater number of vials to be produced in a single production batch.

Radiostabilisers protect radio-labelled compound(s) from radiolysis and therefore lower or prevent a drop in the purity of the radio-labelled compound(s) over their shelf life. While a radiostabiliser may be included to inhibit degradation reactions, such as redox processes, by trapping highly-reactive free radicals, such as oxygen-containing free radicals arising from the radiolysis of water, the enhanced drying step of the present invention has been found to be highly effective and also not have any impact on quantification analysis on the yielded product.

Accordingly, the present invention enables the reaction to be carried out at a higher starting activity, thereby allowing the production of a greater number of patient doses per batch, with a low level of radioimpurities, for example, radioimpurity B. Prior to the invention the reaction had to be carried out with a lower starting activity, in order to control the amount of radioimpurity generated, thereby resulting in fewer product doses per batch.

It will be readily understood by those persons skilled in the art that the embodiments of the invention described herein are capable of broad utility and application. Accordingly, while the invention is described herein in detail in relation to the exemplary embodiments, it is to be understood that this disclosure is illustrative and exemplary of embodiments and is made to provide an enabling disclosure of the exemplary embodiments. The disclosure is not intended to be construed to limit the embodiments of the invention or otherwise to exclude any other such embodiments, adaptations, variations, modifications and equivalent arrangements. The scope of the invention is defined by the appended claims.

1. A method of preparing an [¹⁸F] radiolabelled compound, wherein the method comprises:
(a) an initial drying step comprising evaporating water and acetonitrile from a solution comprising [¹⁸F] fluoride;
(b) a further drying step (fluoride activation step) comprising azeotropic distillation of water from said solution comprising [¹⁸F] fluoride with acetonitrile; and
(c) labelling a precursor compound with [¹⁸F] fluoride from the solution comprising [¹⁸F] fluoride yielded from step (b) to obtain an [¹⁸F] radiolabelled compound;
wherein water content during labelling step (c) that originates from the solution comprising [¹⁸F] fluoride after drying step (b) is less than 500 ppm; and
wherein the water content during labelling step (c) that originates from the precursor compound is no more than 2000 ppm.

2. The method according to claim 1, wherein at least two cycles of the azeotropic distillation with acetonitrile are carried out.

3. The method according to claim 1, wherein three cycles of azeotropic distillation with acetonitrile are carried out.

4. The method according to claim 1, wherein the water content during the labelling step that originates from the solution comprising [¹⁸F] after drying step (b) (the fluoride activation step) is less than 400 ppm.

5. The method according to claim 4, wherein the water content during the labelling step that originates from the solution comprising [¹⁸F] after drying step (b) (the fluoride activation step) is less than 350 ppm.

6. The method according to claim 1, wherein the water content during the labelling step that originates from the precursor compound is no more than 1500 ppm.

7. The method according to claim 6, wherein the water content during the labelling step that originates from the precursor compound is between 500 ppm and 1000 ppm.

8. The method according to claim 1, wherein the total water content during the labelling step is less than 2500 ppm.

9. The method according to claim 8, wherein the total water content during the labelling step is less than 1000 ppm.

10. The method according to claim 1, wherein the solution comprising [¹⁸F] fluoride is eluent from an ion exchange resin, for example, an anion solid phase extraction cartridge.

11. The method according to claim 1, wherein the [¹⁸F] radiolabelled compound obtained in step (c) subsequently undergoes purification steps.

12. The method according to claim 1, wherein the [¹⁸F] radiolabelled compound is a [¹⁸F] fluorine-labelled radiopharmaceutical, or a pharmaceutically acceptable salt thereof.

13. The method according to claim 12, wherein the [¹⁸F] fluorine-labelled radiopharmaceutical, or pharmaceutically acceptable salt thereof, is selected from the group consisting of [¹⁸F] FDG (2-deoxy-2-[¹⁸F] fluoro-D-glucose), [¹⁸F] FMAU (2′-deoxy-2′-[¹⁸F] fluoro-5-methyl-1-beta-D-arabinofuranosyluracil), [¹⁸F] FMISO (¹⁸F Fluoromisonidazole), [¹⁸F] FHBG (9-(4-[¹⁸F] Fluoro-3-[hydroxymethyl] butyl) guanine), [¹⁸F] FES (16a-[¹⁸F] fluoro-17b-estradiol) [¹⁸F] AV-45, [¹⁸F] AV-19, [¹⁸F] AV-1, [¹⁸F] Flutemetamol, [¹⁸F] Flurpiridaz, [¹⁸F] K5, [¹⁸F] HX4, [¹⁸F] W372, [¹⁸F] VM4-037, [¹⁸F] CP¹⁸, [¹⁸F] ML-10, [¹⁸F] T808, [¹⁸F] T807, 2-[¹⁸F] fluoromethyl-L-phenylalanine, GE-135 [¹⁸F] Fluciclatide, GE-212, and GE-226.

14. The method according to claim 12, wherein the [¹⁸F] fluorine-labelled radiopharmaceutical is [¹⁸F] flurpiridaz, or a pharmaceutically acceptable salt thereof having a formula:

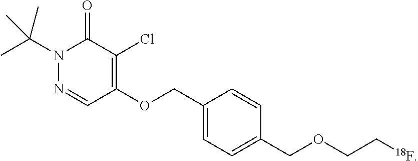

15. The method according to claim 1, wherein starting activity is greater than 100 GBq.

16. The method according to claim 15, wherein the starting activity is 200 to 350 GBq.

17. The method according to claim 1, wherein an amount of radioimpurity B is less than 3.5%.

18. The method according to claim 1, wherein the method is carried out carried out on an automated synthesis system.
